(19) United States
(12) Patent Application Publication
(10) Pub. No.: US 2010/0054996 A1
(43) Pub. Date: Mar. 4, 2010

(54) MICROCHIP FOR PROTEIN FIXATION

(75) Inventors: Hyun Jik Oh, Gyeonggi-do (KR); Tae Joong Yoon, Gyeonggi-do (KR); Sang Hoon Lee, Gyeonggi-do (KR)

Correspondence Address: BERLINER & ASSOCIATES, 555 WEST FIFTH STREET, 31ST FLOOR, LOS ANGELES, CA, 90013, US

(73) Assignee: SEOULIN BIOSCIENCE CO., LTD., Seoul (KR)

(21) Appl. No.: 12/373,877

(22) PCT Filed: Jul. 19, 2007
(86) PCT No.: PCT/KR2007/003499

§ 371 Date: Sep. 17, 2009

(30) Foreign Application Priority Data
Jul. 20, 2006 (KR) 10-2006-0067708

(51) Int. Cl. G01N 33/53

(52) U.S. Cl. 422/68.1

(57) ABSTRACT

Disclosed is a microchip for protein fixation, which fixes a specific protein, such as albumin, immunoglobulin, transferrin, or keratin, to beads so as to detect a micro amount of proteins during a micro analysis process, such as Liquid Chromatography/Mass Spectrometry (LC/MS) or Matrix Assisted Laser Desorption/Ionization Time Of Flight Mass Spectrometry (MALDI-TOF/MS). The microchip for protein fixation reduces the amount of a sample, shortens a reaction time, heightens the reliability in results, and simplifies a working process. For this reason, the beads provided with an antibody attached thereto are contained in a chamber of the microchip having a stratified structure using an organic polymer, i.e., dimethylsiloxane (PDMS), the pressure of a micro amount of a sample passing through the chamber is uniformly distributed onto all the beads, and the micro amount of the sample passes through the beads having a large surface area and is smoothly discharged to the outside at the optimum speed so as to effectively fix the specific protein. Thus, the microchip for protein fixation optimizes the speed of the sample passing through the microchip, and increases an efficiency of fixing the specific protein to the antibody due to the sufficient surface area of the fine-sized beads, thereby obtaining a rapid reaction result.

### TECHNICAL FIELD

The present invention relates to a microchip for protein fixation, and more particularly to a microchip for protein fixation, which selectively fixes albumin, immunoglobulin, or etc.

### BACKGROUND ART

As well known, equipment for analyzing a micro amount of a sample, such as Liquid Chromatography/Mass Spectrometry (LC/MS) or Matrix Assisted Laser Desorption/Ionization Time Of Flight Mass Spectrometry (MALDI-TOF/MS), has been developed now. However, during a process for analyzing a micro amount of proteins, it is difficult to detect a micro amount of proteins due to specific proteins, such as albumin, immunoglobulin, transferrin, keratin, etc.

In order to solve the above problem, dye chromatography is carried out before LC/MS or MALDI-TOF/MS is carried out so as to fix the above-described specific proteins. However, this method has a disadvantage in that proteins to be analyzed may be fixed also.

In order to complement the above disadvantage, an immunochromatograhpic method is carried out. This method immunologically fixes specific proteins using antibodies bonded to these proteins, thus allowing a micro amount of target proteins to be analyzed.

However, in such an immunochromatograhpic method, a sample is refined using a column, and thus the column must be washed after one test and then reused. Accordingly, this method is troublesome and requires a long time to analyze proteins. Further, after the method is carried out, proteins may be attached to antibody resin, and, in the case that the column is repeatedly used, the reliability of results measured by a subsequent analysis process, such as LC/MS or MALDI-TOF/MS, may be lowered.

Further, the immunochromatograhpic method requires at least 250 [symbol] of a sample and thus cannot be used in the case that the amount of the sample is excessively small, and requires a reaction time of 20˜30 minutes and thus cannot obtain a result promptly. Moreover, the immunochromatograhpic method uses resin having an amount of 1.8 times as much as that of the sample.

### DISCLOSURE OF INVENTION

### Technical Problem

Therefore, the present invention has been made in view of the above problems, and it is an object of the present invention to provide a microchip for protein fixation, which shortens a reaction time, heightens the reliability in results, and simplifies a working process.

### Technical Solution

In accordance with an aspect of the present invention, the above and other objects can be accomplished by the provision of a microchip for protein fixation, in which beads provided with an antibody attached thereto are contained in a chamber of the microchip having a stratified structure using an organic polymer, i.e., poly-dimethylsiloxane (PDMS), the pressure of a micro amount of a sample passing through the chamber is uniformly distributed onto all the beads, and the micro amount of the sample passes through the beads having a large surface area and is smoothly discharged to the outside at the optimum speed so as to effectively fix a specific protein.

### Advantageous Effects

Thus, the microchip for protein fixation of the present invention optimizes the speed of the sample passing through the microchip, and increases an efficiency of fixing a specific protein to the antibody attached to the beads due to the sufficient surface area of the fine-sized beads, thereby obtaining a rapid reaction result.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a microchip for protein fixation in accordance with the present invention;

FIG. 2 is a plan view of the microchip in accordance with the present invention, from which a cover glass is stripped;

FIG. 3 is a perspective view of the microchip in accordance with the present invention, from which the cover glass is stripped;

FIG. 4 is a schematic view illustrating the operating state of the microchip in accordance with the present invention; and

FIG. 5 is a schematic view illustrating the state of a sample, which goes around a bead of the microchip in accordance with the present invention.

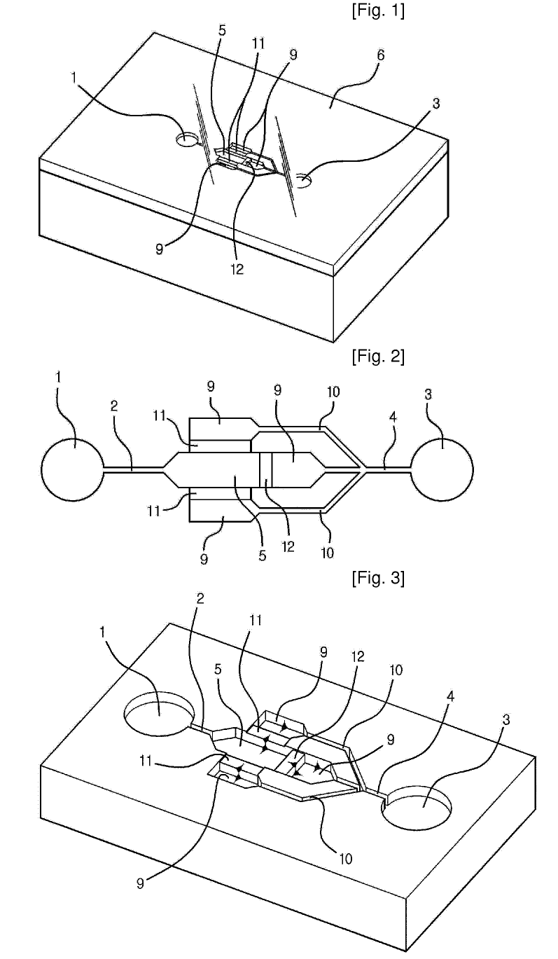

### BEST MODE FOR CARRYING OUT THE INVENTION

Now, the present invention will be described in detail with reference to the annexed drawings.

A microchip for protein fixation in accordance with the present invention is manufactured by soft lithography such that polydimethylsiloxane (PDMS) is poured onto a frame, formed on a silicon wafer using a semiconductor processing technique, such as etching, so as to form channels, a chamber, separation walls, etc., having a desired structure.

The microchip for protein fixation of the present invention is processed such that a chamber 5 is formed between an inlet 1 and an inlet channel 2 and an outlet 3 and an outlet channel 4, and a cover glass 6 covers the above parts so as to form a discharge space 8 having a height smaller than the diameter of beads 7 between the upper ends of separation walls 11 and 12 around the chamber 5 and the cover glass 6 at the front portion and both side portions of the chamber 5 except for the inlet 1. Reservoirs 9 are selectively formed in the discharge space 8 in the above three portions, and are connected to the outlet channel 4 by traveling channels 10.

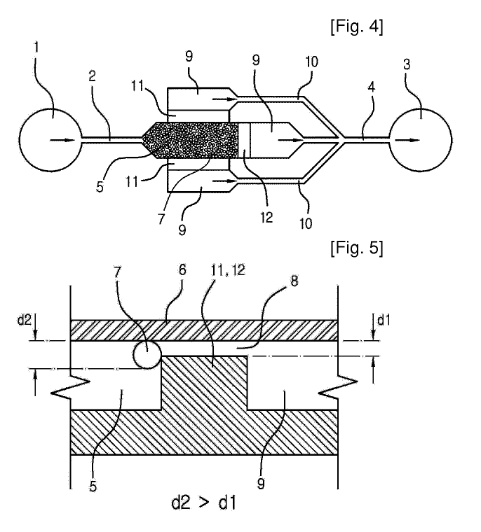

In the microchip for protein fixation of the present invention, a micro amount of a sample enters the inlet 1, passes through the inlet channel 2, and reaches the chamber 5. Since the chamber 5 is filled with a plurality of beads 7, as shown in FIG. 4, the sample flows along the surfaces of the beads 7. Here, a fluid pressure due to the sample is applied to the surfaces of the beads 7. Since the chamber 5 is filled with the beads 7, a high pressure is applied to the beads 7 in directions in which the sample can flow out. Here, in the present invention, the beads 7 are pushed in the directions in which the sample can flow out, i.e., the forward and both sideward directions. However, the height (d1) of the discharge space 8 between the separation walls 11 and 12 and the cover glass 6 is smaller than the diameter (d2) of the beads 7, as shown in FIG. 5, and thus it is possible to prevent the beads 7 from being released from the chamber 5. Thus, the sample can pass gaps between the beads 7 having a circular shape, and be supplied to the reservoirs 9 through the separation walls 11 and 12. The sample collected in the reservoirs 9 is supplied to the outlet channel 4 through the traveling channels 10, and is discharged to the outlet 3. Since the beads 7 are dispersed to the side separation walls 11 and the separation wall 12 in the traveling direction during the above process, it is possible to prevent the beads 7 from being crowded at one side and thus being deformed or damaged due to an excessively high pressure or prevent the sample from being confined due to the crowd of the beads 7, thereby being capable of inducing a stable and effective reaction. In the present invention, the beads 7 has a small diameter of 100 [symbol] or less, and thus has a large surface area. Accordingly, an antibody is attached to the sufficiently large surface areas of the beads 7.

Therefore, a specific protein of the sample, which contacts the surfaces of the beads 7 and then is discharged through the above-described process, can be bonded to the antibody attached to the surfaces of the beads 7. Thus, the specific protein of the sample is effectively reacted with the antibody, is rapidly collected, and then is discharged to the outside. The microchip for protein fixation of the present invention includes small-sized internal structures installed therein such that a micro amount of the sample can continuously passes through the structures. Thus, even when the micro amount of the sample passes through the structures of the microchip, an effectively sufficient reaction can be obtained. In the present invention, the height of the separation walls 11 and 12 of the channels according to the diameter of the beads 7 and the number of the beads 7 in the chamber 5 need to be adjusted. Further, in the present invention, a plurality of structures is etched into a silicon wafer, thus repeatedly mass-producing a plurality of microchips for protein fixation.

The reservoirs 9 may be selectively installed. The reservoirs 9 are installed between the separation walls 11 and 12 and the traveling channels 10, and serve to stably refine and discharge the sample transferred by a micro pump and a micro pipette.

In the present invention, an antibody attached to the beads 7 can be diversified.

That is, the antibody attached to the beads 7 may be anti-albumin antibody, anti-transferrin antibody, anti-Ig G antibody, or anti-keratin antibody.

Although not described in the present invention, since the specific protein fixed to the beads 7 is arbitrarily selected according to purpose, the antibody attached to the beads 7 may be changed suitable to the concrete purpose.

### INDUSTRIAL APPLICABILITY

As described above, the microchip for protein fixation of the present invention uses no more than 10 [symbol] of a sample and thus obtains a refined production, from which a specific protein is removed, while the conventional immunochromatograhpic method requires about 250 [symbol] of a sample, and selectively catches a desired specific protein according to the kind of an antibody attached to beads having a sufficient surface area and thus can be selectively used for various purposes. Further, the microchip for protein fixation of the present invention greatly shortens a reaction time to 30-50% of that of the conventional immunochromatograhpic method so as to obtain a rapid reaction result, and reduces the ratio of the sample and the beads to 10:1, which is 1/18of that of the conventional column method. Moreover, the microchip for protein fixation of the present invention is mass-produced by soft lithography and then has a low production cost, and thus is economical although the microchip is thrown away after use one time.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

1. A microchip for protein fixation, wherein a chamber is formed between an inlet channel formed at one side of an inlet and an outlet channel formed at one side of an outlet, a cover glass covering the inlet, inlet channel, the outlet, the outlet channel so as to form a discharge space having a height smaller than the diameter of beads between the upper ends of separation walls around the chamber and the cover glass at the front portion and both side portions of the chamber except for the inlet, and the chamber is connected to the outlet channel by traveling channels.

2. The microchip for protein fixation according to claim 1, wherein reservoirs are installed between the separation walls and the traveling channels, and are connected to the outlet channel by traveling channels.

3. The microchip for protein fixation according to claim 1, wherein an antibody attached to the beads is anti-albumin antibody.

4. The microchip for protein fixation according to claim 1, wherein an antibody attached to the beads is anti-transferrin antibody.

5. The microchip for protein fixation according to claim 1, wherein an antibody attached to the beads is anti-Ig G antibody.

6. The microchip for protein fixation according to claim 1, wherein an antibody attached to the beads is anti-keratin antibody.
